(19) United States
(12) Patent Application Publication
(10) Pub. No.: US 2014/0195170 A1
(43) Pub. Date: Jul. 10, 2014

(54) APPARATUS, SYSTEM AND METHODS FOR ASSESSING DRUG EFFICACY USING HOLISTIC ANALYSIS AND VISUALIZATION OF PHARMACOLOGICAL DATA

(71) Applicant: General Electric Company, Schenectady, NY (US)

(72) Inventors: Gopal Avinash, Waukesha, WI (US); Ananth Mohan, Waukesha, WI (US); Zhongmin Lin, Waukesha, WI (US); Rick Wascher, Waukesha, WI (US)

(73) Assignee: General Electric Company, Schenectady, NY (US)

(21) Appl. No.: 14/151,383

(22) Filed: Jan. 9, 2014

Related U.S. Application Data
(62) Division of application No. 13/096,046, filed on Apr. 28, 2011.
(60) Provisional application No. 61/386,876, filed on Sep. 27, 2010.

(51) Int. Cl. G01N 33/15

(52) U.S. Cl. CPC G01N 33/15
USPC 702/19

(57) ABSTRACT

Certain examples provide systems and methods for holistic viewing to provide comparative analysis and decision support in a drug development process. An example method includes a computer-implemented method for assessing drug efficacy, comprising: accessing a first data set related to the performance of a target drug for a given indication; accessing a second data set related to a control for the indication; comparing the data for the target drug and the data for the control on at least one of a plurality of different metrics using a holistic analysis, wherein the at least one metric corresponds to an outcome associated with the indication and generating a corresponding report. An example apparatus/system includes a holistic analysis and viewing system to support the assessment of drug efficacy, said system comprising: a standardizer to at least one of standardize and normalize data related to drug development; a deviation analyzer to analyze said data based on at least one of a plurality of different efficacy metrics, wherein a quantified variation between a first data set of results corresponding to an identified target drug and a second data set of results corresponding to a control, wherein said first data set of results is provided for comparison with the second data set of results and the deviation therebetween is compared to the at least one efficacy metric.

### RELATED APPLICATIONS

The present application claims, as a divisional, the benefit of priority of U.S. patent application Ser. No. 13/096,046, filed on Apr. 28, 2011, which claims the benefit of priority to U.S. Provisional Patent Application No. 61/386,876, filed on Sep. 27, 2010, entitled “SYSTEMS AND METHODS FOR HOLISTIC ANALYSIS AND VISUALIZATION OF PHARMACOLOGICAL DATA”, both of which are herein incorporated by reference in their entirety.

### FEDERALLY SPONSORED RESEARCH OR DEVELOPMENT

[Not Applicable]

### MICROFICHE/COPYRIGHT REFERENCE

[Not Applicable]

### BACKGROUND

Pharmaceutical drug development involves significant initial investment for a lengthy development and testing cycle resulting in a new compound coming to market between two to twelve years after initial discovery. Drug development typically includes a plurality of phases including exploratory research, a research phase, a pre-clinical research and development phase, a clinical research and development phase, a product registration and approval phase, and (possibly) a post-marketing phase after the compound is available for sale.

Drug development involves a large amount of data and analysis and evaluation of a compound's effect on a subject in pre-clinical studies and clinical trials. A plurality of sample populations and/or interactions may be tested under a variety of conditions. Resulting pre-clinical and clinical data are integrated into a new drug application (NDA) for submission to a regulatory agency, such as the Food and Drug Administration (FDA).

In general, the drug “discovery” and commercialization of drug products can be summarized as follows: (The various subheadings are intended to suggest or otherwise indicate definable steps or aspects of the overall process.)

### Exploratory Research

### Hit Identification

The hypothesis is validated using biochemical methods and in vivo testing to ensure that the scientific approach is relevant to the disease of interest. The relevant biology is investigated and drug starting points identified.

### Research Phase

### Lead Discovery and Lead Optimization

The initial molecules are further tested in a wider range of biochemical and other models in order to establish that the lead compounds have the potential to become a drug. The lead molecules are further optimized and characterized to determine how to produce the best possible candidate drug. During this time, animal models may be developed to reflect the disease in man as closely as possible to test the compound.

### Pre-Clinical Phase

### Manufacturing

Manufacturing.

The manufacturing process for the new drug is initiated and developed to produce it in sufficient quantities for pre-clinical testing and clinical trial purposes. The new drug must be ready for full manufacture before the start of Phase III trials. This phase continues throughout development.

Pre-Clinical Development.

Pre-clinical development begins before clinical trials or testing in humans may begin and during which important safety and pharmacology data are collected. The main goals of pre-clinical studies are to determine the new drug's pharmacodynamics, pharmacokinetics, ADME and toxicity using blood and tissues. Further pre-clinical development may continue as the new drug progresses through clinical trials.

Application for Investigational New Drug

An application for an IND is made to the FDA, EMEA and/or other regulatory agencies for permission to administer a new drug to humans in clinical trials.

### Clinical Development

Phase I

Phase I trials are conducted primarily to determine how the new drug works in humans, its safety profile and to predict its dosage range. It typically involves between fifty and one hundred healthy volunteers. A pre-marketing strategy may have been instigated as early as Phase I trials to ensure that the market's needs are incorporated into the new drug's overall development, but more usually during the later phases when clinical results are promoted at international symposia in order to develop an awareness amongst the medical community who will ultimately be prescribing the new product. A sales force will be trained and will begin an intense sales and marketing campaign prior to launch.

Phase II

Phase II trials test for efficacy as well as safety and side effects in a group of between one hundred to three hundred patients with the condition for which the new drug is being developed.

Phase III

Phase III trials involve a much larger group of patients, between several hundred and several thousand, which will help determine if the new drug can be considered both safe and effective. It will usually involve a control group using standard treatment or a placebo as a comparison.

### Product Registration and Approval

Marketing and Launch

A pre-marketing strategy may have been instigated as early as Phase I trials to ensure that the market's needs are incorporated into the new drug's overall development, but more usually during the later phases when clinical results are promoted at international symposia in order to develop an awareness amongst the medical community who will ultimately be prescribing the new product. A sales force will be trained and will begin an intense sales and marketing campaign prior to launch

New Drug Application (NDA)

When a product is considered safe and effective from Phase III trials, it must be authorized in each individual country before it can be marketed. All data generated about the molecule is collected and submitted to the regulatory authorities in the US (FDA), European Union (EMEA) and Japan (PMDA) and other countries which may require their own national approvals.

Phase IV Trials

Phase IV trials are conducted after a new drug has been granted a license, approved and launched. In these studies, the new drug is prescribed in an everyday healthcare environment using a much larger group of participants (two to five thousand patients). This enables new treatment uses for the new drug to be developed, comparisons with other treatments for the same condition to be made, and determination of the clinical effectiveness of the new drug in a wider variety of patient types, and more rare side effects, if any, may be detected.

Pharmaceutical medicine uses all the scientific and clinical knowledge acquired by physicians in medical school and postgraduate training—combined with additional regulatory and business skills—to provide a challenging and rewarding career. Unlike clinical medicine, pharmaceutical medicine is part of an industry with huge up front investments for rewards that may or may not come years later. To develop new drugs takes a very, very long time—2 to 12 years from discovery to market, on average—and the cost is extremely high. It costs about $1.8 billion to take a new compound to market and success is quite limited. Only one in 10,000 compounds ever reach the market. Of those only one in three ever recaptures its development costs. High risk indeed! Drug development is a scientific endeavor that is highly regulated because of legitimate public health concerns. Therefore, what is needed is a method and apparatus for streamlining the entire process enabling reproducible and reliable results at every step. The present invention helps provide this capability.

The pre-clinical phase represents bench (in vitro) and then animal testing, including kinetics, toxicity and carcinogenicity. In the U.S., an investigational new drug application (IND) is submitted to the Food and Drug Administration seeking permission to begin the heavily regulated process of clinical testing in human subjects. The clinical research (IND) phase—representing the time from beginning of human trials to the new drug application (NDA) submission that seeks permission to market the drug—is by far the longest portion of the drug development cycle and can last from 2 to 10 years.

Phase I trials, sometimes called, “first in human” trials, are generally conducted on relatively small groups (typically 10 to 30) of healthy volunteers (except for oncology drugs or other potentially toxic compounds) in specialized units resembling small hospitals with 20 to 50 monitored beds. The “inpatient” portion of Phase I trials usually lasts from a day or two to a week (though follow up can last up to about a month), and are designed to assess the safety of a compound and study its pharmacokinetics (Pk—what the body does to the drug) and pharmacodynamics (Pd—what the drug does to the body).

In some cases, human metabolism can differ markedly from animals so that a drug with a half-life of a few hours in dogs may turn out to have a half-life of several days in humans, or a compound with no animal toxicity may cause elevation in liver functions or a prolongation of QT interval in humans. A rough idea of the maximum safe or tolerated dose, as well as a general side effect profile is obtained during Phase I trials.

Many compounds never make it past Phase I, as they are found to have unacceptable side effects. Assuming a compound is shown to be safe for healthy subjects and survives Phase I, then development proceeds to a series of Phase II trials. These trials typically enroll anywhere from about 20 or 30 patients up to a few hundred at most. These patients usually have a relatively “pure” form of the disease for which the drug is intended. In other words, they suffer from as little other intercurrent disease as possible, and the list of concomitant medications they can be taking is usually restricted. For example, patients with newly diagnosed, but untreated, diabetes, with no evidence of end organ damage, would be used to test a new antidiabetic agent.

Phase II trials tend to last only a few weeks to, at most, a few months. Initial Phase II trials (sometimes called, IIa) are pilot trials to determine dose range. They tend to be conducted at specialized centers, like university medical centers, by specialized investigators, such as medical school faculty. Subsequent Phase II trials (often called, IIb) are aimed at elucidating dose response relationships, safety and, for the first time, efficacy, of the compound treating the disease or condition for which it is intended.

Drug interactions are also studied carefully during Phase II as well as Pk and Pd in diseased patients, which can sometimes differ markedly from what was observed in healthy volunteers. Phase II can encompass anywhere from a few to 20 or more clinical trials, and the “development plug” can be pulled—and frequently is—after any of them. Once again, assuming the drug shows sufficient evidence of efficacy and no major safety concerns—whether purely from drug effect, or from drug interactions—a go/no go decision will be made to proceed to Phase III.

Phase III is where the “rubber meets the road.” At least two pivotal Phase III trials demonstrating efficacy and safety in large numbers of patients, including special populations with all forms of the disease or condition to be treated, who may be on multiple other medications, are required for regulatory approval in the U.S. Few drugs have been approved with data from less than two pivotal trials, and, if so, generally require post-marketing commitments to ensure that safety and efficacy is validated after marketing. These trials are randomized, usually placebo-controlled (unless it would be unethical to use a placebo), and often involve an active comparator. They are conducted by less specialized investigators in countries all over the world. Thousands of patients may be enrolled and trials can cost a sponsor $50 million to $100 million each. In addition to the two successful pivotal Phase III trials needed before an NDA can be filed, numerous additional special trials are usually demanded by regulatory agencies throughout the course of the IND clinical development period encompassing Phases I through III.

A few examples of special trials would be to evaluate: Special populations, Renal insufficiency, Hepatic insufficiency, Elderly vs. young, Lactating women, and others/Examples of interactions include: Food or liquids, other drugs used in same indication, Drugs interfering with metabolism, or protein binding, Drugs or substances modifying pharmacodynamic response (e.g., alcohol, sedatives), Drugs or substances which prolong cardiac repolarization, i.e., QT interval (currently an FDA ‘hot button” after withdrawal of several drugs for safety concerns). Similarly, a few examples of “Special conditions” include: Effects on driving automobiles or operating machinery, Effects on performing activities, requiring alertness or concentration, Effects on psychometric or psychological testing, Effects of abrupt drug withdrawal. Some examples of “special toxicities” include: Ocular, Ototoxicity, Rhabdomyolysis, Allergy/Anaphylaxis, Hormonal (e.g., prolactin), Cardiovascular, (QT prolongation), Addiction potential, and more.

If the pivotal trials prove efficacy (usually by meeting or exceeding a predefined statistical “p-value” for a primary efficacy endpoint) and safety, and none of the special trials requested by regulatory agencies uncovers any serious problems, then all data—pre-clinical and clinical—is compiled into an NDA for submission to regulatory agencies.

The NDA includes an integrated summary of efficacy (ISE) and of safety (ISS). It is not unusual for an NDA to run several hundred thousand pages and be delivered to the FDA for regulatory review in one or more large trucks. When evaluating NDAs, regulatory agencies look at: Validity of pivotal studies, Replicability of pivotal studies, (consistency across studies), Generalizability across populations (demographic groups, concomitant medications, intercurrent diseases, geographic regions, and even cultural groups), Establishment of supportable dosage and dose regimen(s), Clinical relevance of efficacy results, Clinical seriousness of safety profile (in context of seriousness of condition being treated), Overall usefulness of drug (risk/benefit ratio).

In the U.S., the FDA does not actually approve the drug itself for sale. It approves the labeling—the package insert. United States law requires truth in labeling, and the FDA assures that a drug claimed to be safe and effective for treatment of a specified disease or condition has, in fact, been proven to be so. All prescription drugs must have labeling, and without proof of the truth of its label, a drug may not be sold in the United States.

The FDA takes, on average, about a year to review a typical, non-expedited NDA, give or take a few months. It may approve the proposed labeling, approve modified labeling, send the sponsor back to conduct additional special, or even pivotal trials, or may refuse approval outright (though, usually it will warn the sponsor if that is likely, giving them an opportunity to withdraw the NDA). Sometimes the FDA will give conditional approval but require additional post-marketing trials to answer specific additional efficacy or safety questions. In addition to mandated conditional regulatory approval or post-marketing surveillance trials, other reasons sponsors may conduct post-marketing trials include: Comparing with competitors (prove non-inferiority or superiority), Widening population (pediatric), Changing formulation or dose regimen (antihypertensive-diuretic combination, or new extended drug from the market at any time), Applying a label extension (such as expanding indication).

Even when an NDA is approved unconditionally, regulatory scrutiny of a drug does not end. In most countries, yearly safety reports must be filed with the applicable regulatory agencies as long as a drug remains on the market, and these agencies independently monitor drug safety. If safety concerns arise, the FDA may demand withdrawal of a drug from the market at any time.

Again, what is needed is a method and apparatus for streamlining the entire process enabling reproducible and reliable results at every step. The present invention helps provide this capability.

### BRIEF SUMMARY

Certain examples provide systems and methods for holistic viewing to provide comparative analysis and decision support in a drug development process. An example method includes accessing data related to drug development; pre-processing the data to prepare the data for measurement and analysis; and analyzing the data based on at least one of a plurality of different metrics. Each metric corresponds to a quantified variation between a first data set of results corresponding to a category in the drug development process. The first data set of results is provided for comparison with a second data set of results corresponding to at least one other category in the drug development process. At least some of the plurality of metrics are aggregated to generate a visual representation representing an integrated comparative visualization for the identified category.

An example holistic analysis and viewing system to support pharmaceutical drug development includes a standardizer, a deviation analyzer, and an output. The standardizer is to process (e.g., standardize and/or normalize, etc.) data related to drug development. The deviation analyzer is to analyze the data based on at least one of a plurality of different metrics. Each metric corresponds to a quantified variation between a first data set of results corresponding to an identified category in the drug development process. The first data set of results is provided for comparison with a second data set of results corresponding to at least one other identified category in the drug development process. The output is to aggregate at least some of the plurality of metrics to generate a visual representation representing an integrated comparative visualization for the identified category. The integrated comparative visualization is to enable a user to observe an outcome represented by at least some of the plurality of different metrics considered collectively to generate a visual report.

An example tangible computer-readable storage medium includes executable instructions for execution using a process. The instructions, when executed, provide a holistic analysis and viewing system to support a drug development process. The system includes a standardizer, a deviation analyzer, and an output. The standardizer is to process (e.g., standardize and/or normalize, etc.) data related to drug development. The deviation analyzer is to analyze the data based on at least one of a plurality of different metrics. Each metric corresponds to a quantified variation between a first data set of results corresponding to an identified category in the drug development process. The first data set of results is provided for comparison with a second data set of results corresponding to at least one other identified category in the drug development process. The output is to aggregate at least some of the plurality of metrics to generate a visual representation representing an integrated comparative visualization for the identified category. The integrated comparative visualization is to enable a user to observe an outcome represented by at least some of the plurality of different metrics considered collectively to generate a visual report.

The present invention can be summarized in a variety of ways including a computer-implemented method for assessing drug efficacy, comprising: accessing a first data set related to the performance of a target drug for a given indication; accessing a second data set related to a control for the indication; comparing the data for the target drug and the data for the control on at least one of a plurality of different metrics using a holistic analysis, wherein the at least one metric corresponds to an outcome associated with the indication and generating a corresponding report.

A quantified variation data set can be generated from the difference between the first and second data sets, wherein the variation data corresponds to an identified category of drugs. In the preferred method, analyzing said variation based on at least one of a plurality of group metrics, wherein each group metric corresponds to the identified category of drugs and including said variation in the report is also desired. Similarly, aggregating at least some of said plurality of metrics to generate a visual representation to generate a visual report is preferred.

The at least some of said plurality of group metrics may be aggregated to generate a visual representation enabling a user to observe an outcome represented by at least some of said plurality of different group metrics and generating a visual report of the visual representation and displaying a report. The plurality of efficacy metrics include a pharmacodynamics metric and a pharmacokinetics metric to model clinical design to

The present inventions may also be defined as a holistic analysis and viewing system to support the assessment of drug efficacy, said system comprising: a standardizer to at least one of standardize and normalize data related to drug development; a deviation analyzer to analyze said data based on at least one of a plurality of different efficacy metrics, wherein a quantified variation between a first data set of results corresponding to an identified target drug and a second data set of results corresponding to a control, wherein said first data set of results is provided for comparison with the second data set of results and the deviation therebetween is compared to the at least one efficacy metric. An output corresponding to the deviation is preferred.

At least some of said plurality of efficacy metrics are used to generate a visual representation of an integrated comparative visualization for the deviation of target drug and control with respect to at least one of the efficacy metrics. In addition, a user interface is also provided to accept user input regarding selection of a class best matching said comparative visualization data with respect to the at least one efficacy metrics.

In a preferred embodiment, the second data set of results comprises placebo test results and the first data set of results comprises target drug test results and wherein at least one of said plurality of efficacy metrics is a separation metric to visualize a separation between placebo results and target drug results. One or more visualization time views for longitudinal analysis of said first or second data sets is desired.

### BRIEF DESCRIPTION OF SEVERAL VIEWS OF THE DRAWINGS

FIG. 1 is a block diagram of an example system to analyze normalized pharmaceutical test or trial data.

FIG. 2 illustrates a flow diagram for an example data mining and learning machine analysis flow.

FIG. 3 illustrates a flow diagram for an example holistic viewer-enabled analysis flow.

FIG. 4 illustrates a flow diagram for an example method for drug classification using a holistic viewer.

FIG. 5 illustrates an example generic depiction of a holistic data classification interface.

FIG. 6 shows a more specific example of a classification interface.

FIG. 7 depicts an example interface to provide holistic views and clustering for a plurality of patients.

FIG. 8 depicts example time-based views provided for longitudinal analysis.

FIG. 9 illustrates an example pharmacokinetic curve using in holistic viewing and analysis.

FIG. 10 illustrates an example holistic view of drug reference parameters over a plurality of test runs using a continuous coded representation for visualization.

FIG. 11 is a block diagram of an example processor system that can be used to implement the systems, apparatus and methods described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EXAMPLES

Although the following discloses example methods, systems, articles of manufacture, and apparatus including, among other components, software executed on hardware, it should be noted that such methods and apparatus are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example methods, systems, articles of manufacture, and apparatus, the examples provided are not the only way to implement such methods, systems, articles of manufacture, and apparatus.

When any of the appended claims are read to cover a purely software and/or firmware implementation, at least one of the elements in an at least one example is hereby expressly defined to include a tangible medium such as a memory, DVD, CD, Blu-ray, etc. storing the software and/or firmware.

Certain examples provide holistic analysis and visualization of pharmacological data. Certain examples provide holistic visualization and analysis of local features extracted from user-selected clinical regions of interest. Certain examples provide holistic data visualization and related applications in a pharmacological viewer.

A holistic approach to data, such as pharmacological data, can be used to bring diverse types of data together in one application for viewing and analysis. A holistic view and analysis can be used as part of a pharmaceutical testing and drug delivery process. The holistic view and analysis can be used to replace and/or supplement a data mining approach.

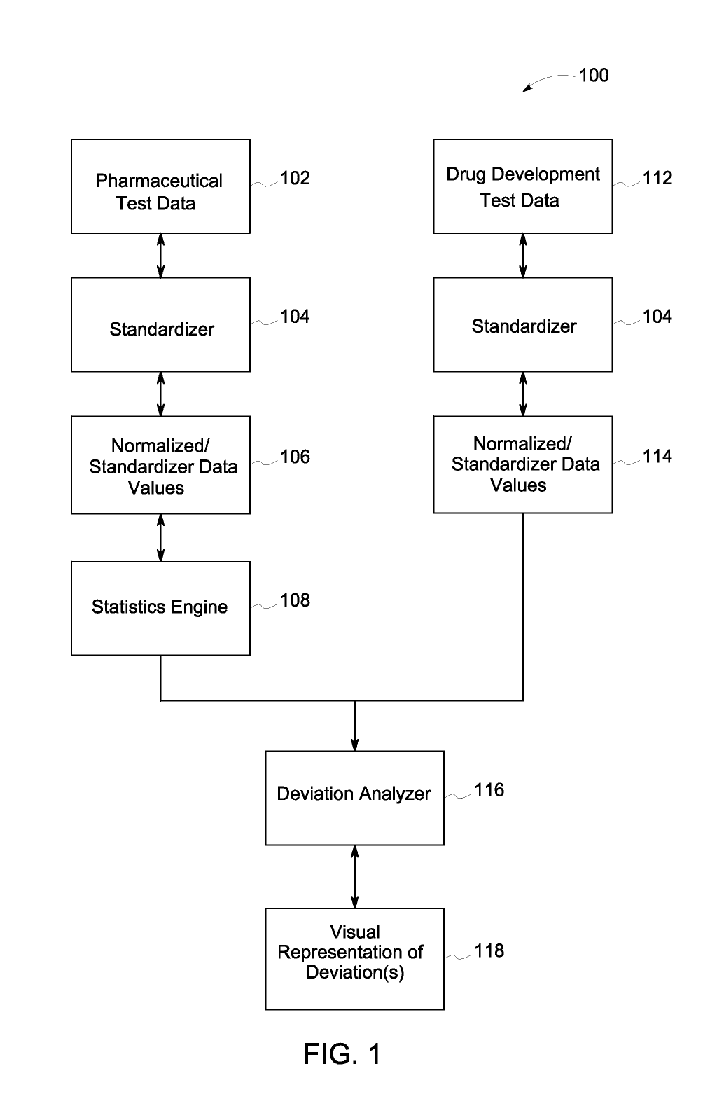

FIG. 1 is a block diagram of an example system 100 to analyze normalized pharmaceutical test or trial data. The system 100 gathers pharmaceutical data and creates descriptors that define a normal state or result which can be used to identify abnormal states and/or varying results in one or more chemical compounds, patients, test subjects, and/or other research/trial conditions, for example.

The system 100 includes pharmaceutical test data 102 with respect to a “normal”, control, reference, or expected value. The normal pharmaceutical test data 102 is acquired from one or more tests or projections involving drug compounds, test subjects, etc., identifying desired effects, concentrations, limitations, etc., in a proposed drug.

The pharmaceutical test data 102 is received by a standardizer 104 that normalizes and/or standardizes the pharmaceutical test data 102, thus generating normalized and/or standardized pharmaceutical data 106 of a plurality of normal subjects. The system 100 also includes a statistics engine 108 that determines statistics 110 of the normalized and standardized metadata 106 of the normal subjects. The statistics engine 108 operates on the normalized and/or standardized metadata 106 of each pharmaceutical test. The system 100 creates descriptors that define a normal, reference, or control state that can be used to identify abnormal states/results in drug development data.

The system 100 includes drug development test data 112 and/or other data related to a pharmaceutical drug development process. The drug development test data 112 is received by a standardizer 104 that normalizes and/or standardizes the drug development test data 112, thus generating normalized and/or standardized drug development test data 114.

In certain examples, data 106 and/or 112 can be standardized and normalized for one or more subjects. Then, an average of the data is determined. A database or other data set of control and/or reference data, for a particular matched subject/criteria group, can be created. The data set can be criterion(a) specific and include mean and standard deviation data for normal/expected/control subject data sets. A well-defined “normal” cohort can be used to create a data set of normal/control/reference data. The set of normal cohort are clinically tested to determine the normal data information. In the standardized space, each label can be assigned a mean value and associated standard deviation based on the data samples from the cohort of normal cases. Drug development test data can be similarly standardized and normalized. Thereafter, a comparison of each of number of labels in the normalized subject data set and the drug development test data set is performed. A visual output of the comparison is generated.

Thus, a normal/reference/control/expected data set can be created using a standardization/normalization transformation of individual data values pertaining to all labels in all axes. In addition, a statistical metric can be established that is used to determine individual label-based abnormalities. A deviation from a reference, control, or expected vale can be displayed in a visual manner to facilitate a holistic view of result(s).

The system 100 also includes a deviation analyzer 116 that determines deviation(s) 118 between the reference or control statistic(s) 110 and the drug development test data 114 for each pharmaceutical test.

In an example, deviation(s) between data sets can be determined according to the following equation:

$$Δ {a}_{i} = \frac{{α}_{i} - {μ}_{ai}}{{σ}_{ai}} . \qquad Equation 1$$

In Equation 1, αᵢ is the i^{th} label of axis “a” and μ_{αi} and σ_{αi}. Equation 1 is applied to all the labels in all the axes and the resultant is a deviation data “vector”. Equation 1 is also known as the Z-score, standard score, or normal score, for example.

In certain examples, to determine deviation(s) in pharmacological data, available data is converted to a common unit of measurement (e.g., by the standardizer 104). Where the data being analyzed is represented in various units of measurement, determining a deviation includes converting the data to one particular unit of measurement in order to avoid a mathematically invalid deviation.

In certain examples, a deviation analysis includes label value-by-label value comparison of each clinical-test label in the drug development data to a corresponding clinical-test label in the comparison of the drug development test data and the control or reference subject data. Each clinical-test label belongs to a clinical category in the drug development test data, for example.

In certain examples, a deviation data vector is determined that describes how far the drug development test data deviates from the data to which it is being compared.

An output, such as a display, can generate a visual graphical representation of the deviation(s) 118 for each of the pharmaceutical test(s). Thus, system 100 helps identify and determine drug characteristics, drug effects, drug dosage, patient impact, and/or other data relevant to pharmaceutical drug development when compared against a cohort of normal controls using a structured approach based on a comprehensive data.

In certain examples, a visual representation of deviation for each drug development test provides drug development evaluation in a holistic and visual form. Deviation data can be displayed in a consistent and visually acceptable sense that may allow for improved drug development as the information is presented to the visual cortex of the brain for pattern matching rather than the memory recall based on computer-generated data mining.

One illustrative example is that all the data is ordered in a consistent from (ordering using clinical relevance is best) where the rows represent the axes and the columns represent each label within that axis. Each active pixel of this graph is assigned a color from a color scale that maps the deviation value of the label to a conspicuous concern value. A practitioner can see a pattern of deviation in conjunction with a relative degree of concern in one snapshot for a variety of axes and data. The visual depiction helps allow for a more rapid and consistent evaluation, for example.

In certain examples, visualization of data deviation includes generating Z-score table representations of the drug development deviation data. A table format representation shows a deviation from normal/control/reference and Z-scores. The table can be represented in graphical image format as well to provide a snap-shot of all deviation data for quick review to identify abnormal conditions/results.

In certain examples, rather than comparing drug development test results to reference, control, or expected values, one group or cohort of drug development test results is compared to another group of drug development test results to visualize conformity(ies) and/or deviation(s) between the two sets of test results.

Certain examples can identify variations in available data, such as pharmaceutical drug development data, and allow a user to visualize the data with respect to a reference (such as using the system 100 above). Using visualization of data deviation, a user does not need to be an expert to see deviation from a normal or reference value as an indication of an abnormal result.

In certain examples, drug development data and associated processing/analysis can be color-coded and/or otherwise differentiated to help a user visualize areas that are different from “normal”, expected, or reference value(s). Patterns, such as concentrations or “hot spots”, in the data can be quickly visualized and appreciated by a user, for example. Additionally, in certain examples, while patterns and/or abnormalities can be visualized, other details are not lost when displaying available data to a user.

In certain examples, a view of drug development data over time can be provided. A view can provide a representation of longitudinal trends in the data over time. For example, a deviation in one patient or test subject's longitudinal trends from a reference population or cohort can be tracked and visualized over time.

In certain examples, a distribution (e.g., one time and/or longitudinal over time) of drug data can be processed and visualized by taking a group of patients, candidates, etc., and comparing the group as a whole. Characteristics such as drug characteristics, disease signatures, symptoms, side effects, etc., can be viewed to determine how they deviate from a control group. Patterns identified from these view(s) can be fed back into the drug development process, for example. Characteristics of a reference versus a target can be visualized and evaluated on an individual and/or group basis, for example.

For pharmacological analysis, each metric examined can compare target data to a reference, for example. A plurality of metrics can be combined and presented in a single report. An analysis can be conducted any phase of the drug development process. For example, potential clinical trial or study candidates can be identified via a holistic visualization and review. Subject responses from candidates can also be reviewed and analyzed. Clinical trial results can be processed and visually depicted for user review. In addition or group or population-based analysis, drug compound test data, drug characteristics, etc., can be visually depicted and analyzed with respect to a reference or control, for example. In certain examples, data mining applied in pharmaceutical drug development can be supplemented or replaced by holistic viewing systems and methods described herein.

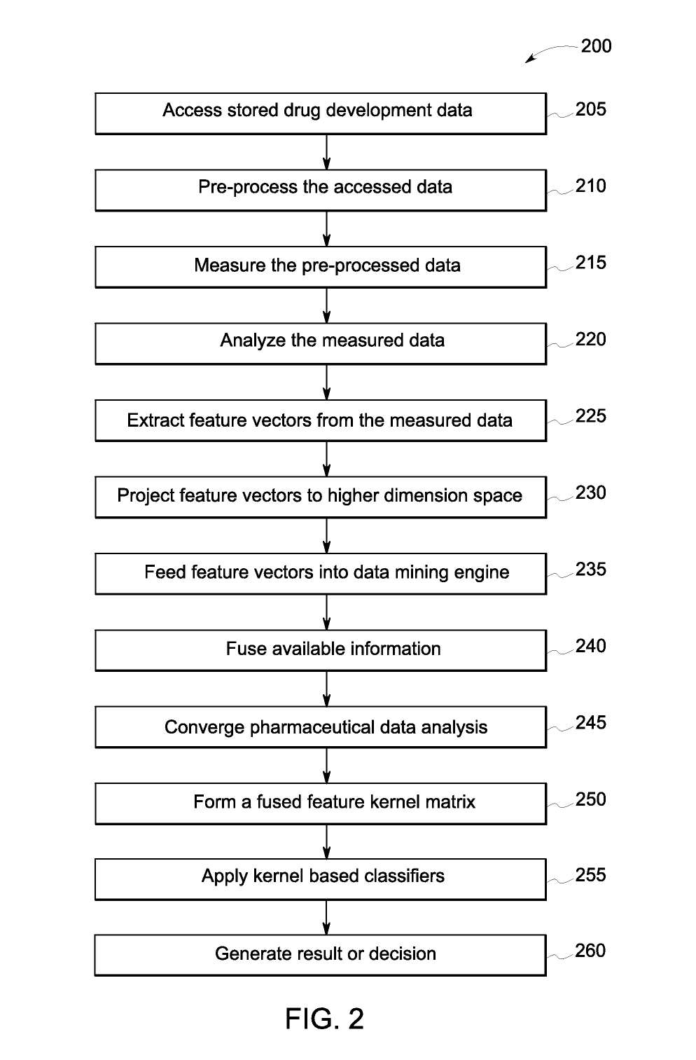

FIGS. 2-4 are flow diagrams representative of example machine readable instructions that may be executed to implement example systems and methods described herein, and/or portions of one or more of those systems (e.g., systems 100 and 1100) and methods. The example processes of FIGS. 2-4 can be performed using a processor, a controller and/or any other suitable processing device. For example, the example processes of FIGS. 2-4 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example processes of FIGS. 2-4 can be implemented using coded instructions (e.g., computer readable instructions) stored on a non-transitory computer readable medium such as a flash memory, a read-only memory (ROM), a random-access memory (RAM), a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example processes of FIGS. 2-4 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example processes of FIGS. 2-4 can be implemented manually or as any combination(s) of any of the foregoing techniques, for example, any combination of firmware, software, discrete logic and/or hardware. Further, although the example processes of FIGS. 2-44 are described with reference to the flow diagrams of FIGS. 2-4, other methods of implementing the processes of FIGS. 2-4 can be employed. For example, the order of execution of the blocks can be changed, and/or some of the blocks described can be changed, eliminated, sub-divided, or combined. Additionally, any or all of the example processes of FIGS. 2-4 can be performed sequentially and/or in parallel by, for example, separate processing threads, processors, devices, discrete logic, circuits, etc.

FIG. 2 illustrates a flow diagram for an example data mining and learning machine analysis flow 200. At 205, stored data associated with pharmaceutical drug development is accessed. For example, data including pharmaceutical compound model(s); pharmacodynamic data; pharmacokinetic data; absorption, distribution, metabolism, and excretion (ADME) data; toxicity data; drug safety profile data; dosage data; side effect data; etc., can be accessed for processing, viewing, and analysis.

At 210, pre-processing is performed on the accessed data. Pre-processing can include, for example, data corrections, selection of one or more subsets of data, normalization of data relative to a reference or threshold, etc. At 215, the data is measured. For example, the pre-processed data is measured to extract quantitative information in relation to one or more of the accessed types of data.

At 220, the measurement data is analyzed. For example, at 225, data analysis can include extracting feature vectors from the measurement data. Thus, a drug can be represented by a vector representative of chemical structure including frequency of small fragments and/or frequency of labeled paths to classify chemical compounds. At 230, analysis can include projecting the feature vectors into a higher dimensional space (e.g., using a support vector machine (SVM)). At 235, analysis can also include feeding the feature vectors into a data mining (DM) engine. At 240, analysis can include fusion of the available information. In certain examples, features can be weighted based on relevant domain knowledge (e.g., knowledge of a pharmaceutical data domain).

At 245, pharmaceutical data analysis is converged. Convergence includes, for example, at 250, forming a fused feature kernel matrix from a plurality of available feature kernels (e.g., feature vector(s), SVM output(s), DM output(s), etc. At 255, kernel-based classifiers (e.g., (SVM, linear discriminant analysis (LDA), principal component analysis (PCA), nearest neighbor (NN), etc.) are applied to the fused feature kernel matrix. In certain examples, a kernel function can be selected based on one or more preferences, parameters, priorities, and/or circumstances to process and obtain a better or more optimal fused kernel. At 260, convergence includes generating a result or decision based on the kernel-based classification.

In certain examples, parameters can be improved or optimized using one or more training algorithms. Training and pharmaceutical test data sets can be separated, for example. Training prior to data mining can help improve selection of the right classifier for the available pharmaceutical data.

However, data mining methods can introduce difficulty when performing integrated quantifiable comparative analysis and decision support during a pharmaceutical drug development process. In addition, automated data mining techniques and applications can provide useful results but are hard to adequately prove in a regulated environment. Automated data mining techniques can also suffer limitations when encountering samples with missing data, noise in the data, and datasets too small for statistical significance or confidence.

Several differences are provided between a holistic viewer (HV) and a data mining or learning machine (DM/LM) approach. For example, a transformation of data differs between data mining and a holistic approach. In DM/LM, the transformation is to a feature vector space. In HV, the transformation is to a homogenizing space, such as a deviation from a reference value.

Training also differs between HV and DM/LM. For example, in DM/LM, training involves manual tweaking parameters of classifiers by a scientist/engineer. In HV, no training is required. Further, no data reduction is needed with a holistic view. In DM/LM, testing is accomplished by a trained classifier engine. In HV, testing is done by having a user understand overall patterns displayed in the data. While an end user in DM/LM only reviews the results, an end user (e.g., a clinician) in HV is directly involved in analyzing results and patterns in the data.

The HV provides output in a visual form, wherein relationships among various variables can be displayed and directly understood by the user. All forms of data are transformed to a common, consistent visual form. A Holistic Viewer approach provides an alternate or supplemental technique that keeps a human user involved and participating in the pharmacological data analysis process.

Certain examples utilize holistic views to visualize abnormality in medical (e.g., pharmaceutical) data by transforming raw results with respect to reference datasets (such as deviation from “normal” cohorts). Individually standardizing and normalizing clinical results enables the concurrent visualization of multi-disciplinary medical data and reveals characteristic disease signatures and abnormality patterns in specific patients or patient populations under review. Using a holistic viewer helps to improve, enhance and further enable comparative analysis during various stages in the development process of a pharmaceutical drug including discovery, clinical development and post-launch activities, for example.

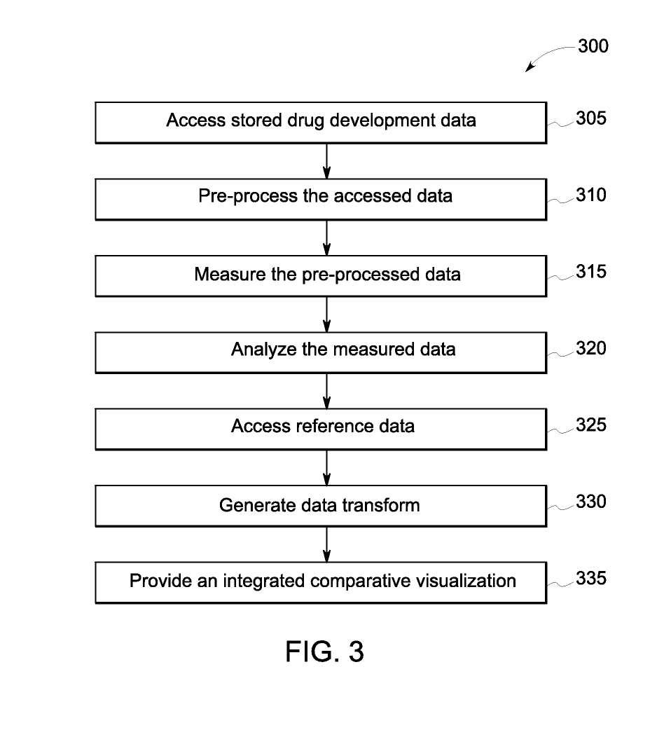

FIG. 3 illustrates a flow diagram for an example holistic viewer-enabled analysis flow 300. At 305, stored data associated with pharmaceutical drug development is accessed. For example, data including pharmaceutical compound model(s); pharmacodynamic data; pharmacokinetic data; absorption, distribution, metabolism, and excretion (ADME) data; toxicity data; drug safety profile data; dosage data; side effect data; etc., can be accessed for processing, viewing, and analysis.

At 310, pre-processing is performed on the accessed data. Pre-processing can include, for example, data corrections, selection of one or more subsets of data, normalization of data relative to a reference or threshold, etc. Pre-processing can leverages data that is fed into data mining and automated analytics processes, for example

At 315, the data is measured. For example, the pre-processed data is measured to extract quantitative information. At 320, the measurement data is analyzed. For example, at 325, data analysis can include accessing reference data (if applicable). At 330, a data transform is generated. For example, a transformation can involve a distribution analysis (e.g., a one-time distribution, a longitudinal distribution over time, etc.), a deviation with respect to a reference, etc.

At 335, an integrated comparative visualization of the analyzed data is provided. For example, a deviation map (e.g., a color-based or “heat” map) of comparative drug development data can be provided to a user for review. Using the data visualization, a user can arrive at result and/or decision, for example. Using a holistic approach to analysis of pharmacological data and visualization of the results helps keep the user involved and aware of a range of test and/or other results, for example.

In certain examples, a visual report is generated by method(s) and/or system(s) for integrated quantifiable comparative analysis and decision support in a pharmaceutical drug development process. The report utilizes and includes a plurality of different metrics. Each metric corresponds to a distinct quantified variation between a first data set of results corresponding to an identified category in the drug development process. The first data set of results is provided for comparison with a second data set of results corresponding to at least one other distinct category. At least some of the plurality of metrics are aggregated to generate a visual representation representing an overall outcome for the identified category. At least some of the plurality of metrics are used to observe an overall outcome represented by the plurality of different metrics when considered collectively to generate the visual report therefrom.

A holistic view can be used at a plurality of stages in a drug development process. For example, a holistic viewer can be applied during drug discovery. Pharmaceutical data classification can be facilitated using the holistic viewer. Holistic classification can be applicable in drug discovery, clinical trials, and/pr product efficacy analysis, for example.

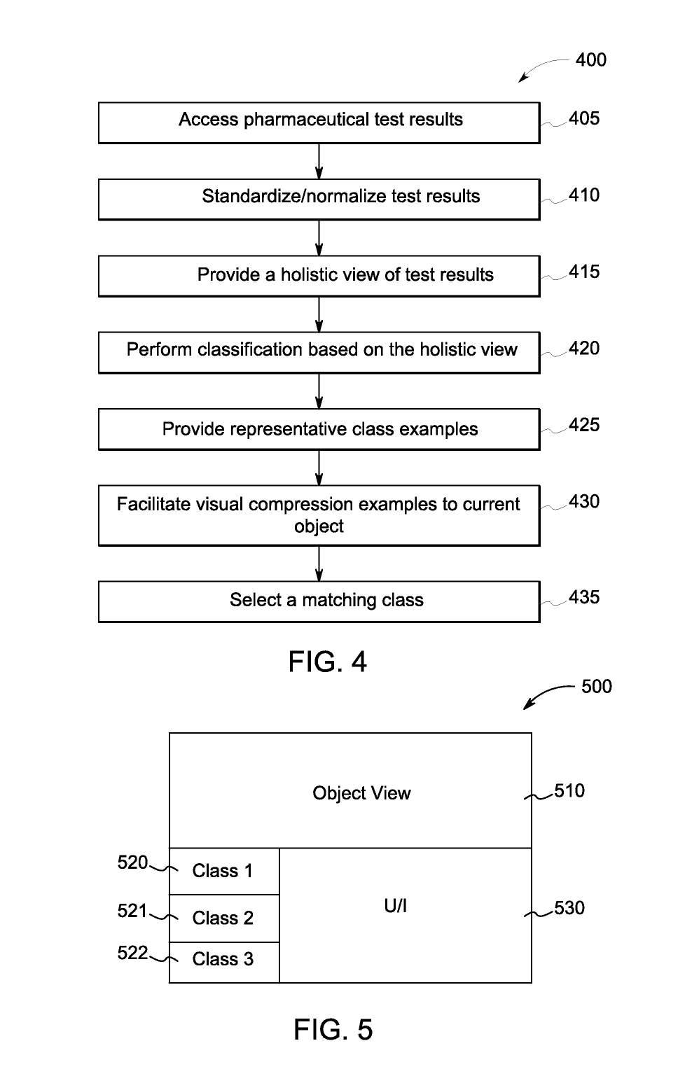

FIG. 4 illustrates a flow diagram for an example method 400 for drug classification using a holistic viewer. At 405, pharmaceutical test results are accessed.

At 410, test results are processed to standardize and/or normalize the data. For example, results can be standardized and/or normalized according to a reference value, threshold, range, etc. At 415, a holistic view of the test results is provided. At 420, classification is performed based on the holistic view.

At 425, representative examples of classes corresponding to each of the desired groups are provided. For example, groups for pharmaceutical cases can include patient cohorts, drugs, tests, disease types, disease severities, etc. For example, classes for a disease type of Alzheimer's disease can include normal, mild cognitively impaired (MCI), Alzheimer's disease, etc. Classes for a drug development can include one or more outcomes, reactions, concentrations, etc.

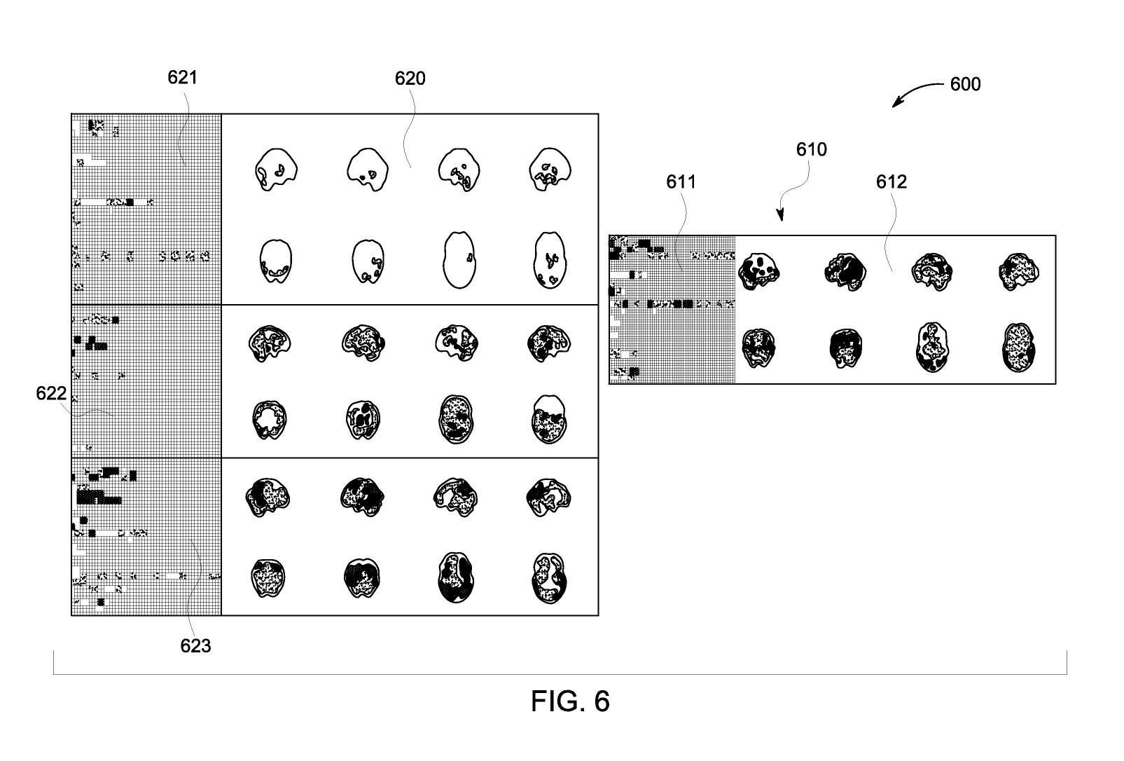

At 430, the representative examples are visually compared with a current object. For example, a generic view 500 is provided in FIG. 5. A specific example view 600 is shown in FIG. 6. At 435, a class of the most matching representative examples is selected as the class of the current object.

FIG. 5 illustrates an example generic depiction of a holistic data classification interface 500. The interface 500 includes an object view 510, one or more classifications 520-522, and a user interface 530. The object view 510 provides a view of available data, which can be compared by a user against one or more classes 520-522 of representative data. The user interface 530 allows a user to manipulate the data, the classifications, and/or provide a diagnosis and/or further instruction, for example. Via the user interface 530, a user can indicate which class 520-522 best fits the data presented in the object view 510.

FIG. 6 shows a more specific example of a classification interface 600. In the interface 600, available clinical 611 and imaging 612 data are shown in an object view 610 window. Available classifications 620 include a normal classification 621, a mild cognitive impairment classification 622, and an Alzheimer's disease classification 623 shown via the interface 600. A user can select an appropriate classification 620 based on clinical information 611, imaging information 612, and/or a combination of clinical and imaging information 611, 612, for example. Similarly, a user can select a drug response based on a view of available data in comparison to classifications of drug responses, disease characteristics, other relevant indicators, etc.

Certain examples can be used to provide clustering using a holistic viewer. Clustering is similar to classification, but there are some differences. For example, a clustering process does not have pre-determined classes but rather has options to create as many ad hoc classes as needed that seem to be related. For example, test results can be grouped together based on one or more pre-determined themes.

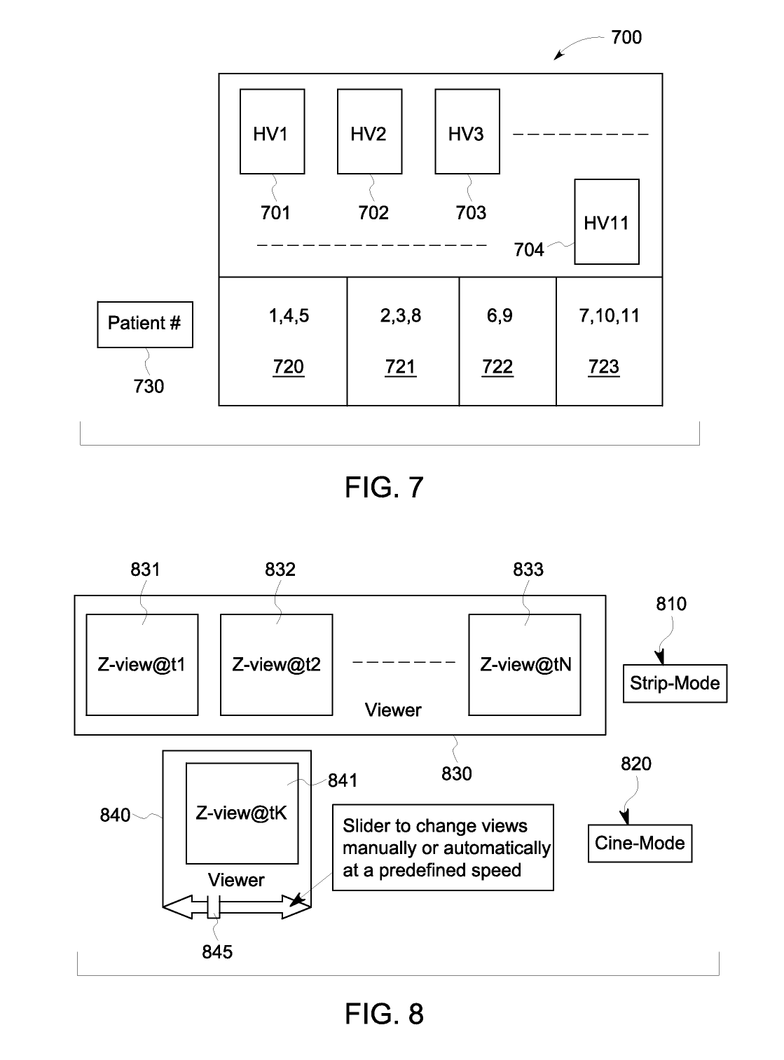

In the example depicted in FIG. 7, an interface 700 provides holistic views 701-704 and clustering 720-723 for a plurality of patients based on patient number 730. Using the interface, a user can cluster holistic views (HVs) 701-704 of eleven (11) patients into four (4) groups 720-723 based on one or more criterion. For example, patients 1, 4, and 5 are in a different cluster 720 than patients 2, 3, and 8. As shown in FIG. 7, HV(s) 701-704 can provide information to draw conclusion(s) and determine further action(s) based on visual depiction of the information and relationship(s) within the information (e.g., patient clustering). Holistic view clustering can also be used for ad hoc grouping of objects including patients, drugs, tests, diseases, severities, etc., during drug discovery and/or clinical trials, for example.

In many cases of drug discovery, tests can be evaluated to determine separation between a placebo and one or more drugs being evaluated. A distribution analysis (e.g., a one-time distribution, a longitudinal distribution over time, etc.) shown via a holistic viewer can be used to visualize placebo/drug distinction(s). The holistic distribution viewer can represent non-numerical data forms in their native data forms, and disease signatures can be obtained for those tests.

For example, a placebo group can be compared to a drug group to evaluate comparative effect. A separation metric shows test results that provide a best separation with imaging and non-imaging tests given patient, drug, and/or other constraints. Results derived from the separation metric and/or other metrics in the comparison can be used as feedback to advance and/or further refine drug development, for example. Characteristics of a placebo versus a drug compound can be visualized and evaluated on an individual and/or group basis, for example.

In certain examples, as depicted in FIG. 8, new time views can be provided for longitudinal analysis. Drug discovery can benefit from novel time trend representations. As shown, for example, in FIG. 8, longitudinal or Z-views can be presented in a “strip mode” 810 and/or a “cine mode” 820. In some examples, these representations can be performed on partial results using, for example, a filter, and/or on an entire data set.

The views 810, 820 shown in FIG. 8 provide alternative presentations of longitudinal data tracked over time. For example, the strip mode view 810 includes a viewer 830 including a plurality of longitudinal data views 831-833 over time. The cine mode view 820 includes a viewer 840 providing a longitudinal data view 841 and a control 845 (e.g., a slider) to change the view 841. The control 845 can be used to change the view 841 manually, automatically at a pre-defined or set speed, etc.

In certain examples, a holistic analysis and view can be applied to pharmacokinetics and/or pharmacodynamics. Pharmacokinetics (PK) characterizes absorption, distribution, metabolism, and elimination properties of a drug. Pharmacodynamics (PD) defines a physiological and biological response to an administered drug. PK/PD modeling establishes a mathematical and theoretical link between these two processes and helps to better predict drug action. Integrated PK/PD modeling and computer-assisted trial design via simulation are being incorporated into many drug development programs and are having a growing impact on drug development and testing.

PK/PD testing is typically performed at every stage of the drug development process. Because development is becoming increasingly complex, time consuming, and cost intensive, companies are looking to make better use of PK/PD data to eliminate flawed candidates at the beginning and identify those with the best chance of clinical success.

An analysis of PD/PK includes determining a maximum drug concentration (Cmax), a time to maximum concentration (Tmax), a minimum drug concentration or remains (Cmin), etc. For different drug components, interaction with a human body can be different. Multiple “runs” can be performed using one or more attributes including 1) across compound candidates, 2) across compound type, 3) across time, 4) in target disease affected organs, 5) in body distribution, 6) in specific organs that might be hurt, etc.

For example, a holistic viewer can be used for drug interaction studies. A goal of the interaction study is to determine whether there is any increase or decrease in exposure to a substrate in the presence of an interacting drug. If there is an interaction, implications of the interaction are assessed by understanding PK/PD relations. As an example, a holistic viewer can be used to figure out salient experimental runs by analyzing and visualizing the parameters with respect to one or more references. Parameters to analyze can include time-to-maximum (Tmax), maximum concentration (Cmax), average concentration, residual time, remains (Cmin), area under curve (AUC), etc. Drug exposure, expressed in terms of AUC (area under a drug plasma concentration-time curve), Cmax (maximum drug concentration in plasma), and/or an alternative parameter, for example, can be related to drug dose level and associated toxicological outcomes. Based on toxicokinetic data at a no-observed toxic effect dose, an acceptable exposure limit in humans can be defined.

Cmax indicates a maximum or “peak” concentration of a drug observed after its administration. Cmin represents a minimum or “trough” concentration of a drug observed after its administration and just prior to the administration of a subsequent dose. For drugs eliminated by first-order kinetics from a single-compartment system, Cmax, after n equal doses given at equal intervals can be represented by C0(1−fn)/(1−f)=Cmax, and Cmin=Cmax−C0, for example.

An area under a plot of plasma concentration of drug (not a logarithm of the concentration) against time after drug administration is represented by AUC. The area can be determined by the “trapezoidal rule”, for example. According to the trapezoidal rule, data points are connected by straight line segments; perpendiculars are erected from the abscissa to each data point; and the sum of the areas of the triangles and trapezoids so constructed is computed. When the last measured concentration (Cn, at time tn) is not zero, the AUC from tn to infinite time is estimated by Cn/kel. An elimination rate constant (kel) is a first order rate constant describing drug elimination from the body. Kel is an overall elimination rate constant describing removal of the drug by all elimination processes including excretion and metabolism. The elimination rate constant is the proportionality constant relating the rate of change drug concentration and concentration or the rate of elimination of the drug and the amount of drug remaining to be eliminated, for example.

The AUC is of particular use in estimating bioavailability of drugs, and in estimating total clearance of drugs (ClT). Following single intravenous doses, AUC=D/CIT, for single compartment systems obeying first-order elimination kinetics; alternatively, AUC=C0/kel. With routes other than the intravenous, for such systems, AUC=F·D/CIT, where F is the bioavailability of the drug. The ratio of the AUC after oral administration of a drug formulation to that after the intravenous injection of the same dose to the same subject can be used during drug development to assess a drug's oral bioavailability, for example.

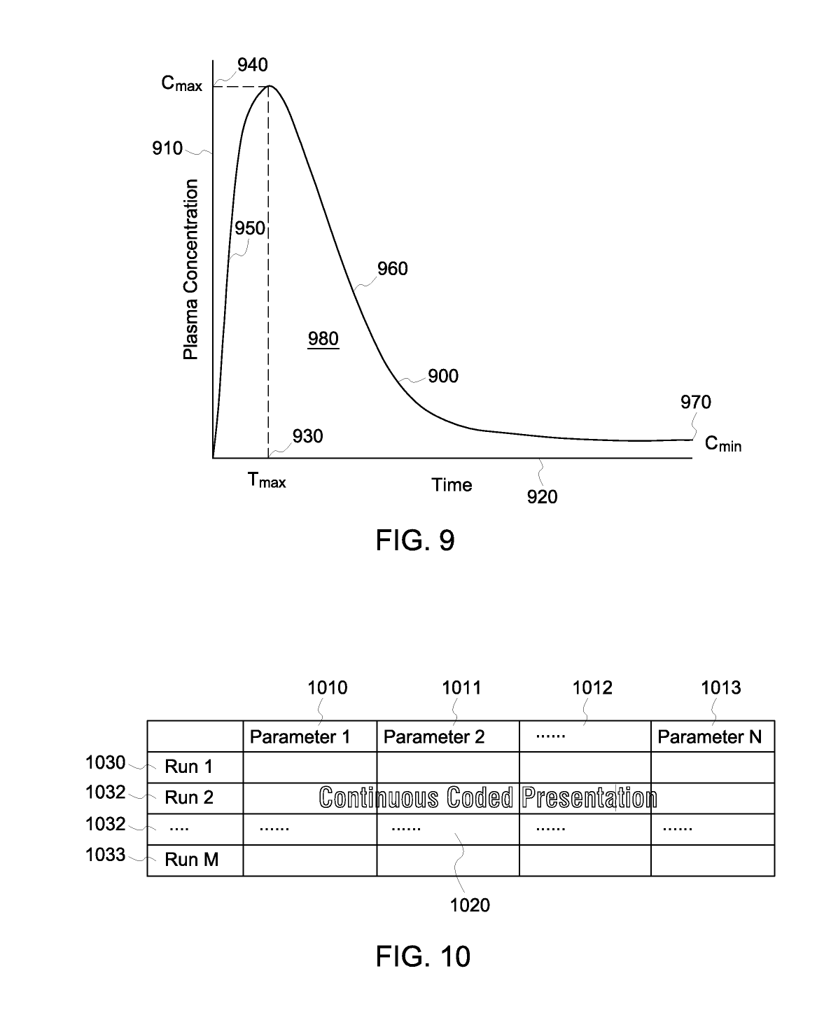

FIG. 9 illustrates an example PK curve 900 including parameters discussed above. As shown on the graph of FIG. 9, the curve 900 is plotted based on plasma concentration 910 versus time 920. At a time to maximum (Tmax) 930, a maximum concentration (Cmax) 940 is identified. Prior to achieving Cmax 940 at Tmax 930, a drug is in an absorption phase 950 in a patient. After Tmax 930, the drug is in an elimination phase 960 resulting in a drug residue or remains (Cmin) 970. Based on this information, an area under the curve (AUC) 980 can be determined.

Holistic views can be created in a number of different ways. As illustrated, for example, in FIG. 10, a drug can be selected as a reference to analyze one or more parameters 1010-1014 of different drug interaction with the body including time-to maximum, maximum concentration, area under curve, and remains. The parameters 1010-1014 can be presented as a continuous color coded representation 1020 for easy visualization, for example. The parameters 1010-1014 can be evaluated over multiple test runs 1030-1034, for example. Along with the drug development and clinical trial, the reference drug and key parameter(s) can be updated for a next round clinical trial and drug improvement, for example. A preferred or “ideal” candidate can be picked by visual comparison and/or by an appropriate criterion (e.g., a weighted score), for example.

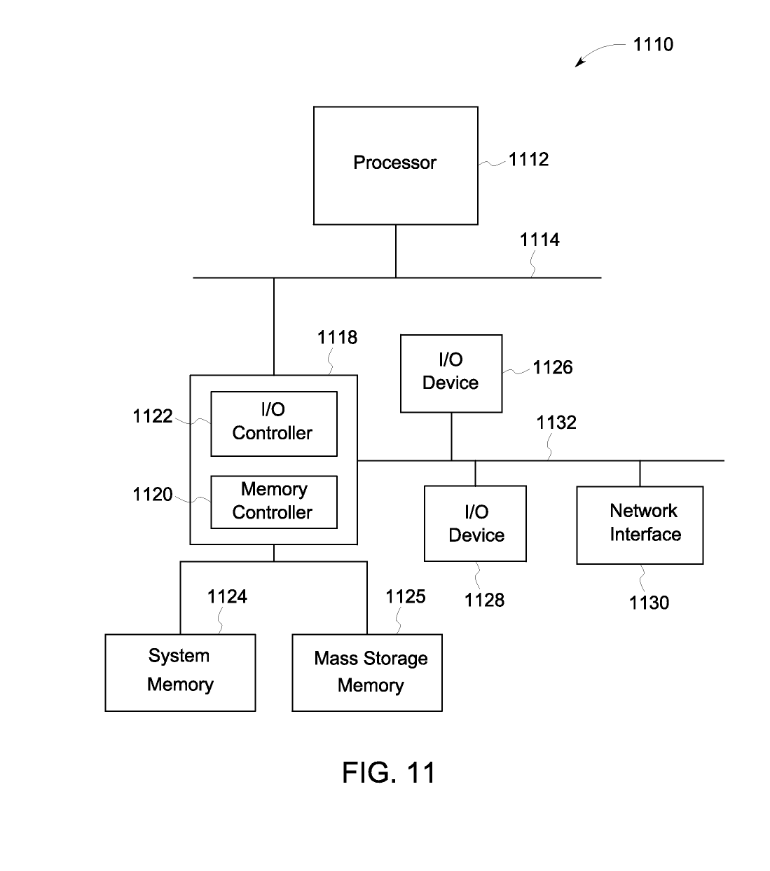

FIG. 11 is a block diagram of an example processor system 1110 that can be used to implement the systems, apparatus and methods described herein. As shown in FIG. 11, the processor system 1110 includes a processor 1112 that is coupled to an interconnection bus 1114. The processor 1112 can be any suitable processor, processing unit or microprocessor. Although not shown in FIG. 11, the system 1110 can be a multi-processor system and, thus, can include one or more additional processors that are identical or similar to the processor 1112 and that are communicatively coupled to the interconnection bus 1114.

The processor 1112 of FIG. 11 is coupled to a chipset 1118, which includes a memory controller 1120 and an input/output (I/O) controller 1122. As is well known, a chipset typically provides I/O and memory management functions as well as a plurality of general purpose and/or special purpose registers, timers, etc. that are accessible or used by one or more processors coupled to the chipset 1118. The memory controller 1120 performs functions that enable the processor 1112 (or processors if there are multiple processors) to access a system memory 1124 and a mass storage memory 1125.

The system memory 1124 may include any desired type of volatile and/or nonvolatile memory such as, for example, static random access memory (SRAM), dynamic random access memory (DRAM), flash memory, read-only memory (ROM), etc. The mass storage memory 1125 may include any desired type of mass storage device including hard disk drives, optical drives, tape storage devices, etc.

The I/O controller 1122 performs functions that enable the processor 1112 to communicate with peripheral input/output (I/O) devices 1126 and 1128 and a network interface 1130 via an I/O bus 1132. The I/O devices 1126 and 1128 may be any desired type of I/O device such as, for example, a keyboard, a video display or monitor, a mouse, etc. The network interface 1130 may be, for example, an Ethernet device, an asynchronous transfer mode (ATM) device, an 802.11 device, a DSL modem, a cable modem, a cellular modem, etc. that enables the processor system 1110 to communicate with another processor system.

While the memory controller 1120 and the I/O controller 1122 are depicted in FIG. 11 as separate blocks within the chipset 1118, the functions performed by these blocks may be integrated within a single semiconductor circuit or may be implemented using two or more separate integrated circuits.

Thus, certain examples provide holistic visual systems, methods, and apparatus to process drug development data related to target and reference value(s) according to one or more metrics and provide output to a user for visual review and analysis. Conformity and/or deviation between a group of test data and a reference/control data set and/or another group of test data can be graphically provided to a user for holistic analysis, rather than a numerical result provided by computer data mining. For example, drug development and clinical trial data can be compared to reference drug and parameter data to better facilitate and/or adjust a next of clinical trial and drug improvement. Certain examples provide an additional technical effect of dynamic metric identification and data analysis to provide an integrated comparative visualization of an available body of drug development data to enable a user to arrive at a result and/or make a decision regarding a next step in a drug development process.

Certain examples contemplate methods, systems and computer program products on any machine-readable media to implement functionality described above. Certain examples can be implemented using an existing computer processor, or by a special purpose computer processor incorporated for this or another purpose or by a hardwired and/or firmware system, for example.

One or more of the components of the systems and/or steps of the methods described above may be implemented alone or in combination in hardware, firmware, and/or as a set of instructions in software, for example. Certain examples can be provided as a set of instructions residing on a computer-readable medium, such as a memory, hard disk, DVD, or CD, for execution on a general purpose computer or other processing device. Certain examples can omit one or more of the method steps and/or perform the steps in a different order than the order listed. For example, some steps/blocks may not be performed in certain examples. As a further example, certain steps may be performed in a different temporal order, including simultaneously, than listed above.

Certain examples include computer-readable media for carrying or having computer-executable instructions or data structures stored thereon. Such computer-readable media can be any available media that may be accessed by a general purpose or special purpose computer or other machine with a processor. By way of example, such computer-readable media can include RAM, ROM, PROM, EPROM, EEPROM, Flash, CD-ROM, DVD, Blu-ray, optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions comprise, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Certain examples can be practiced in a networked environment using logical connections to one or more remote computers having processors. Logical connections can include a local area network (LAN) and a wide area network (WAN) that are presented here by way of example and not limitation. Such networking environments are commonplace in office-wide or enterprise-wide computer networks, intranets and the Internet and can use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Examples can also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of embodiments of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated computer-readable media provide nonvolatile storage of computer-executable instructions, data structures, program modules and other data for the computer.

While the invention has been described with reference to certain examples or embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A non-transitory computer readable storage medium having stored thereon a computer program comprising instructions, which, when executed by a computer, cause the computer to:
access test result data from a computer memory;
generate analysis of said test result data by at least one if standardizing and normalizing said test result data and by comparing to reference data;
perform medical classification of the test result data based on said analysis to determine comparison examples; and
display to a user a holistic medical view of the test result data and one or more comparison examples.

2. The computer readable storage medium of claim 1, wherein the test result data is generated by performing tests on a patient and recording the test result data into said computer memory.

3. The computer readable storage medium of claim 2, wherein the performed tests are radiological imaging tests.

4. The computer readable storage medium of claim 2, wherein the performed tests are pharmaceutical tests.

5. The computer readable storage medium of claim 1, wherein the test result data is pharmaceutical test data or pharmacological test data.

6. The computer readable storage medium of claim 1, wherein the instructions further cause the computer to:
receive a user selection of a perceived match between the test result data and one of the displayed comparison examples.

7. The computer readable storage medium of claim 6, wherein the instructions further cause the computer to:
record the user selection in the computer memory; and
send the user selection and the test result data to a remote computing device via a network.

8. The computer readable storage medium of claim 1, wherein the holistic medical view displays both clinical information and imaging information of the test result data or the comparison examples.

9. The computer readable storage medium of claim 8, wherein the clinical information is displayed as deviation patterns indicating a plurality of deviation values where the test result data deviates from the reference data.

10. The computer readable storage medium of claim 9, wherein the deviation pattern comprises colors that map the deviation value of the data to a conspicuous concern value.

11. The computer readable storage medium of claim 9, wherein the deviation patterns comprise colors indicating the extent of the distribution shift of the standardized or normalized test result data from the reference data.

12. The computer readable storage medium of claim 8, wherein the instructions further cause the computer to:
receive a user selection of a match between the displayed holistic view of the test result data and one of the displayed comparison examples; and
display a visible query to a user to determine if the user selection was based on the displayed clinical information, imaging information, or both.

13. The computer readable storage medium of claim 1, wherein the instructions further cause the computer to:
receive a user selection of a match between the displayed holistic view of the test result data and one of the displayed comparison examples;
refine the reference data based on the received user selection; and
store the refined resulting data in the computer memory.

14. The computer readable storage medium of claim 1, wherein the holistic medical view of the test result data comprises colors indicating the extent of the distribution shift of the standardized or normalized test result data from the reference data.

15. The computer readable storage medium of claim 1, wherein the reference data are clinical test labels and the comparison includes label value-by-label value comparison of each clinical test label with said test result data.

16. The computer readable storage medium of claim 1, wherein said comparison examples are derived from disease signatures.

17. The computer readable storage medium of claim 1, wherein said all comparison examples displayed are displayed in a consistent visual form to each other and to the holistic medical view of the test result data.

18. The computer readable storage medium of claim 1, wherein at least part of said test result data is generated by performing tests on a plurality of individuals who have consumed a pharmaceutical product.

19. A non-transitory computer readable storage medium having stored thereon a computer program comprising instructions, which, when executed by a computer, cause the computer to:
access pharmaceutical test result data from a computer memory;
generate analysis of said test result data by at least one of standardizing and normalizing said test result data and by comparing to reference data;
perform medical classification of the test result data based on said analysis to determine comparison examples derived from disease signatures;
display to a user a holistic medical view of the test result data and a plurality of comparison examples;
wherein the holistic medical view of the test data comprises deviation patterns indicating a plurality of deviation values where the test result data deviates from the reference data; and
wherein said all comparison examples displayed are displayed in a consistent visual form to each other and to the holistic medical view of the test result data.

20. The computer readable storage medium of claim 19, wherein the deviation patterns comprise colors indicating the extent of the distribution shift of the at least one of standardized and normalized test result data from the reference data.
